(19) United States
(12) Patent Application Publication
(10) Pub. No.: US 2026/0151053 A1
(43) Pub. Date: Jun. 4, 2026

(54) FINGERMARK ENHANCEMENT APPARATUS

(71) Applicant: Foster & Freeman Limited, Evesham (GB)

(72) Inventor: Roberto King, Evesham (GB)

(73) Assignee: Foster & Freeman Limited, Evesham, EN (GB)

(21) Appl. No.: 18/864,240

(22) PCT Filed: May 15, 2023
(86) PCT No.: PCT/GB2023/051265

§ 371 Date: Nov. 8, 2024

(30) Foreign Application Priority Data
May 13, 2022 (GB) 2207029.6

(51) Int. Cl. A61B 5/1172

(52) U.S. Cl. CPC A61B 5/1172

(57) ABSTRACT

A fingermark enhancement apparatus comprising a chamber, the chamber comprising a sealable space for accommodating at least a portion of an object comprising a fingermark. The apparatus further comprising a fluid transfer device for providing at least one fingermark enhancement fluid to the chamber, the fluid transfer device comprising a fluid inlet connectable to a source of fingermark enhancement fluid, and a fluid outlet configured to provide the fingermark enhancement fluid to the chamber, and wherein the fluid transfer device is configured to provide the fingermark enhancement fluid to the chamber in at least one of: gaseous, vapour or aerosol form.

### FIELD OF THE INVENTION

The present invention relates to the enhancement of fingerprints and particularly, but not exclusively, to fingermark enhancement apparatus that can deliver fingermark enhancement fluid, such as reagents or dyes, to an object.

### BACKGROUND TO THE INVENTION

Fingermarks, whether latent or visible, are typically enhanced in some way prior to processing. Known methods of enhancing fingermarks often involve immersing an item of evidence comprising a fingermark in a solution, such as a reagent solution.

Such traditional methods of fingermark enhancement (e.g. “dipping” or “dunk” methods) suffer from drawbacks. The manual process of immersing, removing, rinsing and drying evidence is laborious and time-consuming, and it can take up to 24 hours to process an object of evidence in this way.

For some known enhancement processes, there is the potential for cross-contamination of evidence, particularly of DNA, if the same solution is used for different items of evidence. The solutions are often expensive, and large quantities are required, and therefore the risk of the solution being re-used is thought to be high.

Using large quantities of enhancement solution, which may be hazardous, makes the enhancement process costly, difficult to dispose of waste, non-environmentally friendly and, for some flammable enhancement fluids such as ethanolic BY40 there is an increased safety risk.

The inventors have appreciated the shortcomings in known fingermark enhancement processes.

### STATEMENTS OF INVENTION

According to a first aspect of the present invention there is provided a fingermark enhancement apparatus comprising:
a chamber comprising: a sealable space for accommodating at least a portion of an object comprising a fingermark;
a fluid transfer device for providing at least one fingermark enhancement fluid to the chamber, the fluid transfer device comprising:
   a fluid inlet connectable to a source of fingermark enhancement fluid;
   a fluid outlet configured to provide the fingermark enhancement fluid to the chamber;
      wherein the fluid transfer device is configured to provide the fingermark enhancement fluid to the chamber in at least one of: gaseous, vapour or aerosol form.

The fingermark enhancement apparatus may be a portable apparatus. The fingermark enhancement apparatus may be a portable handheld apparatus. The fingermark enhancement apparatus may be a handheld apparatus or operable by hand, or formed from one or more handheld components. The chamber may be a portable chamber. The chamber may be a handheld chamber. The chamber may be a portable handheld chamber. The fluid transfer device may be portable. The fluid transfer device may be a handheld device. The fluid transfer device may be a portable handheld device. The chamber may be sized to be movable and operable by hand. The fluid transfer device may be sized to be movable and operable by hand.

The fingermark could be visible, latent, full or partial, or any combination thereof. The fingermark may be a processed fingermark, processed using one or more fingermark processing techniques.

The fluid transfer device may be configured to provide the fingermark enhancement fluid to the chamber in gaseous form. The fluid transfer device may be configured to provide the fingermark enhancement fluid to the chamber in vapour form. The fluid transfer device may be configured to provide the fingermark enhancement fluid to the chamber in aerosol form. The fluid transfer device may be configured to provide the fingermark enhancement fluid to the chamber in vapour form or in aerosol form. The fluid transfer device may be configured to provide the fingermark enhancement fluid to the chamber in vapour form, aerosol form, gaseous form, or any combination thereof.

The fluid transfer device may be a nebuliser. The fluid transfer device may be a vaporiser. The fluid transfer device may be an aerosol generator.

The fluid transfer device may comprise a nebuliser, a vaporiser, and/or an aerosol generator. The nebuliser may nebulise the fingermark enhancement fluid. The vaporiser may vaporise the fingermark enhancement fluid. The aerosol generator may generate an aerosol using, at least in part, the fingermark enhancement fluid.

The fluid transfer device may be configured to convert the source of fingermark enhancement fluid to at least one of: gaseous, vapour or aerosol form, or a combination thereof. The fluid transfer device may be configured to convert the source of fingermark enhancement fluid from a liquid to at least one of: gaseous, vapour or aerosol form.

The fluid transfer device may comprise a transducer for converting the source of fingermark enhancement fluid to at least one of: gaseous, vapour, or aerosol form. The transducer may be a piezoelectric transducer. The transducer may be an ultrasonic transducer. The transducer may be an ultrasonic piezoelectric transducer. The transducer may be configured to convert liquid fingermark enhancement fluid to at least one of: gaseous, vapour, or aerosol form.

The fingermark enhancement fluid may be a fingermark enhancement reagent, blood enhancement reagent, fingermark enhancement dye, DNA enhancement reagents, or the like. The fingermark enhancement fluid may include at least one of: one or more fingermark enhancement reagents, one or more blood enhancement reagents, one or more fingermark enhancement dyes, one or more DNA enhancement reagents, or the like.

The fingermark enhancement fluid may be a liquid. The fingermark enhancement fluid may be a solid. The fingermark enhancement fluid may be a gas. The fingermark enhancement fluid may comprise at least one of: a liquid, solid or gas, or any combination thereof.

The fingermark enhancement fluid may comprise a solvent. The solvent may be an aqueous solvent or a non-aqueous solvent. The solvent may be an organic solvent. The organic solvent may be a hydrofluoroether solvent.

The fingermark enhancement fluid may comprise one or more reagents and/or one or more dyes. The one or more reagents may include at least one of: blood enhancement reagents, body fluid sensitive reagents, or the like. The one or more dyes may include DNA stains, or the like. The reagents may be selected from: ninhydrin, DFO, Oil Red O, cyanoacrylate, ruthenium tetroxide (RTX), 1,2-Indanedione, luminol, STK sperm tracker, or the like. The one or more dyes may be selected from BY40, R6G, RAM, Ardrox, Basic Red, or the like.
The apparatus may comprise one or more sources of fingermark enhancement fluid, or two or more sources thereof.

The fluid transfer device may comprise a single fluid inlet, or two or more fluid inlets, the, or each fluid inlet being connectable to a source of fingermark enhancement fluid.

The fluid transfer device may comprise a single fluid outlet, or two or more fluid outlets, the, or each, fluid outlet being configured to provide fingermark enhancement fluid to the chamber.

The fluid transfer device may be configured to provide at least one of: vapourised, aerosol, or gaseous, or combination thereof, fingermark enhancement fluid to the chamber.

The apparatus may comprise a single fluid transfer device, or two or more fluid transfer devices.

The apparatus may comprise a single chamber. The apparatus may comprise a plurality of chambers.

The chamber may be configured such that a fluid flow path exists from the, or each, fluid outlet of the fluid transfer device to the, or each, object when accommodated in the sealable space of the chamber.

The chamber may comprise a base portion. The chamber may comprise one or more walls. The chamber may comprise a top portion.

The chamber may have one or more arcuate wall portions, curved wall portion(s), or the like.

The base portion of the chamber may be a planar base portion, an arcuate-shaped base portion, a curved base portion, a dome shaped base portion, or the like.

The chamber may be a substantially cylindrical member, or a substantially partially cylindrical member, or a substantially dome-shaped member, or a partially dome shaped member. The chamber may take any suitable shape or form.

At least a portion of the chamber may be transparent. The chamber may be at least partially transparent. The chamber may be a transparent chamber. The chamber may be opaque, or non-transparent.

The apparatus may comprise one or more sealing members for sealing the chamber. The sealing member may be a lid, a seal, a hatch, a door, or the like. The chamber may form an enclosed space when sealed.

The chamber may be configured to be sealable when engaged with an object comprising a fingermark. In this arrangement, and for example, the object could be a wall or other structure, having a fingermark thereon, and the chamber could be pressed against the object to seal the chamber. The chamber may be engageable with the object by hand. The chamber may be configured to be pressable against the object by hand.

The chamber may comprise one or more object engagement members for engaging an object comprising a fingermark. The one or more object engagement members may be located at the top portion of the chamber. The one or more object engagement members may be planar surfaces.

The one or more object engagement members may be arranged in a square, rectangular, circular, or elliptical arrangement, or any suitable arrangement. The one or more object engagement members may be arranged at an end portion of one or more walls of the chamber.

The chamber may be configurable between an unsealed and sealed configuration. The chamber may be reversibly sealable between the unsealed and sealed configurations. The chamber may be configurable between the unsealed and sealed configurations by adding the sealing member to the chamber or by bringing the chamber into engagement with an object comprising a fingermark. The chamber may be configurable between the unsealed and sealed configurations by adding the sealing member to the chamber or by bringing the chamber, by hand, into engagement with an object comprising a fingermark.

In the sealed configuration, the chamber may be sealed substantially air-tight. The chamber may be hermetically sealable.

The fluid outlet of the fluid transfer device may be located at one or more walls of the chamber. The fluid outlet of the fluid transfer device may be located at a mid-region of one or more walls of the chamber. The fluid outlet of the fluid transfer device may be located at the base portion of the chamber. The fluid outlet of the fluid transfer device may be located at the base portion or at one or more walls of the chamber.

The, or each, fluid outlet may be arranged to protrude into the chamber.

The apparatus may be configured to disperse the fingermark enhancement fluid within the chamber. The apparatus may be configured to circulate the fingermark enhancement fluid within the chamber. The apparatus may be configured to disperse or circulate the fingermark enhancement fluid within the chamber.

The apparatus may comprise at least one fluid flow device. The fluid flow device may be a fluid dispersion element. The fluid flow device may be a fluid circulation element. The fluid flow device may be configured to increase the flow of the fingermark enhancement fluid within the chamber. The fluid flow device may be configured to disperse, or circulate, the fingermark enhancement fluid within the chamber.

The fluid flow device may be located at least partially within the chamber. The fluid flow device may be located at a lower region of the chamber.

The fluid flow device may be spaced apart from the, or each fluid outlet of the fluid transfer device.

The fluid flow device may be a fan, or the like.

The chamber may be configured to accommodate one or more objects, each of the objects comprising one or more fingermarks. The chamber may be configured to accommodate any suitable number of such objects.

The object may be a planar object, such as a piece of paper or the like. The object may be a porous object or non-porous object. The object may have been pre-treated with an adhesive, such as a cyanoacrylate adhesive.

The chamber may comprise one or more mounting points for mounting, hanging, or locating the object(s) comprising the fingermark(s) at least partially within the chamber. The fluid outlet may be configured to direct the fingermark enhancement fluid generally towards at least one of the mounting points. The one or more mounting points may be located at an upper region of the chamber.

The fingermark enhancement apparatus may be configured to be a lightweight apparatus, which may be transportable by hand.

According to a second aspect of the present invention there is provided a method of enhancing a fingermark, the method comprising the steps of:
(i) providing a fingermark enhancement apparatus comprising:
   a chamber comprising a sealable space for accommodating at least a portion of an object comprising a fingermark;
   a fluid transfer device for providing at least one fingermark enhancement fluid to the chamber, the fluid transfer device comprising:
      a fluid inlet connectable to a source of fingermark enhancement fluid;
      a fluid outlet configured to provide the fingermark enhancement fluid to the chamber;
         wherein the fluid transfer device is configured to provide the fingermark enhancement fluid to the chamber in at least one of: gaseous, vapour or aerosol form,
(ii) locating at least a portion of an object comprising a fingermark in the sealable space of the chamber;
(iii) connecting the fluid inlet to a source of fingermark enhancement fluid; and
(iv) providing the fingermark enhancement fluid to the chamber in at least one of: gaseous, vapour or aerosol form.

It should be appreciated that the order that the steps are recited and any numbering given to those steps in any of the methods or processes described herein is only exemplary, and that the steps may be carried out in any order except where it is clear that a specific order is meant and/or a specific order is required or essential for the proper functioning of the method or process.

Embodiments of the second aspect of the present invention may include one or more features of the first aspect of the present invention or its embodiments. Similarly, embodiments of the first aspect of the present invention may include one or more features of the second aspect of the present invention or its embodiments.

According to a third aspect of the present invention there is provided a system for enhancing a fingermark, the system comprising:
a fingermark enhancement apparatus comprising:
   a chamber comprising a sealable space for accommodating at least a portion of an object comprising a fingermark;
   a fluid transfer device for providing at least one fingermark enhancement fluid to the chamber, the fluid transfer device comprising:
      a fluid inlet connectable to a source of fingermark enhancement fluid;
      a fluid outlet configured to provide the fingermark enhancement fluid to the chamber;
         wherein the fluid transfer device is configured to provide the fingermark enhancement fluid to the chamber in at least one of: gaseous, vapour or aerosol form; and
         a source of fingermark enhancement fluid.
Embodiments of the third aspect of the present invention may include one or more features of the first and/or second aspects of the present invention or their embodiments. Similarly, embodiments of the first and/or second aspects of the present invention may include one or more features of the third aspect of the present invention or its embodiments.

### BRIEF DESCRIPTION OF THE DRAWINGS

Embodiments of the invention will now be described, by way of example, with reference to the drawings, in which:

FIG. 1 shows an embodiment of a fingermark enhancement apparatus in accordance with an embodiment of the invention;

FIG. 2 shows the embodiment of FIG. 1 when in engagement with an object, such as a wall;

FIGS. 3a to 3d show the use of the apparatus to enhance fingermarks;

FIG. 4 shows a split image of fingermarks processed using the apparatus 1 of FIG. 1 (top half) and fingermarks that have been enhanced using a traditional dipping method (bottom half);

FIG. 5 shows another split image of fingermarks processed using the apparatus (bottom half) and using a traditional dipping method (top half); and

FIG. 6 shows an alternative embodiment of the fingermark enhancement apparatus of FIG. 1, in which the chamber is dome shaped.

### DESCRIPTION OF PREFERRED EMBODIMENTS

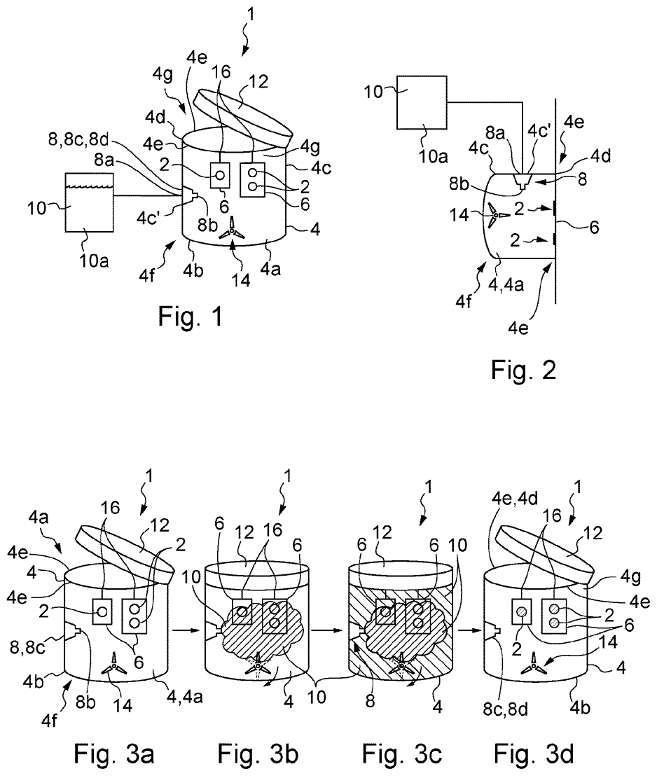

With reference to FIGS. 1 to 6, a fingermark enhancement apparatus 1 is shown. The apparatus 1 of the present invention, unlike traditional methods of fingermark enhancement, can provide fingermark enhancement fluid to an item of evidence in gaseous, vapour or aerosol form, or any combination thereof if desired. Delivering the enhancement fluid in this manner automates the process, reduces waste, mitigates cross contamination, and reduces costs.

The apparatus 1 is suitable for enhancing fingermarks 2, which may be visible/latent, full/partial, or any combination thereof. Other uses of the apparatus 1 will be apparent to those of skill in the art.

The fingermark enhancement apparatus 1 comprises a transparent chamber 4 having a sealable space 4a for accommodating at least a portion of an object 6 comprising a fingermark 2. In the embodiment shown here, the entire object 6 is located within the sealable space 4a, but in other embodiments it may be that only a portion of the object 6 is located within the sealable space 4a.

As shown in FIG. 1, the fingermark enhancement apparatus 1 includes a fluid transfer device 8 for providing at least one fingermark enhancement fluid 10 to the chamber 4. The fluid transfer device 8 comprises a fluid inlet 8a connectable to a container 10a of liquid fingermark reagent or dye solution (an example of a source 10a of fingermark enhancement fluid 10). It will be understood that other sources, and other types, of fingermark enhancement fluid could be used. For example, the fingermark enhancement fluid could include reagents, dyes, or the like, as is known in the field of fingermark processing. In other embodiments, the fingermark enhancement fluid 10 could comprise at least one of: a liquid, solid or gas, or any combination thereof.

In the embodiments illustrated and described here, the fingermark enhancement apparatus 1 is a portable handheld apparatus 1, which as will be described in more detail below, in some embodiments can be easily engaged, by hand, with a structure. The apparatus 1 is lightweight and transportable by hand, and can therefore be easily used at a crime scene. For example, if an upper region of a wall is to be examined using the apparatus 1, then because the chamber 4 and fluid transfer device 8 are handheld and portable, the user can lift the apparatus 1 to any height within reach and use the apparatus 1 to provide fluid to the object 6. Likewise, in embodiments where the chamber 4 is sealable with a lid or the like, the apparatus 1 is easy to use and transport. It will be understood that in other embodiments it may be that only part of the apparatus 1 needs to be portable, such as only the chamber 4 or fluid transfer device 8 thereof, or in other embodiments it need not be portable.

The fluid transfer device 8 also includes a fluid outlet 8b configured to provide the fingermark enhancement fluid 10 to the chamber 4.

In the embodiments illustrated and described here, the fluid transfer device 8 is configured to provide the fingermark enhancement fluid 10 to the chamber 4 in vapour form by vapourising liquid from the container 10a. However, in other embodiments, the fluid transfer device 8 could provide the fingermark enhancement fluid 10 to the chamber 4 in at least one of: gaseous, vapour or aerosol form, as required. For example, the fluid transfer device 8 could have any combination of suitable components to enable the desired output, such as a combination of vapouriser, aerosol generator, and a suitable gas fluid outlet or the like. It will be appreciated that in some embodiments this might mean the apparatus 1 has more than one fluid transfer device 8, or that the fluid transfer device 8 is multi-function.

The desired output of the fluid transfer device 8 may depend on the source 10a of fingermark enhancement fluid 10. Whilst a liquid is used here, in other embodiments a liquid/solid mixture, a gas, or any combination of solid, liquid and/or gas fingermark enhancement fluid may be used, which may determine which type of fluid transfer device 8 should be used. Similarly, the nature of the object 6 and/or the fingermark 2, may determine which of: vapour, gaseous, or aerosol form, or combination thereof, is best to use.

Turning back to FIG. 1, the fluid transfer device 8 comprises a vaporiser 8c for vaporising the fingermark enhancement fluid 10. The vaporiser 8c may take any suitable form. In other embodiments, the fluid transfer device 8 may comprise a nebuliser, a vaporiser 8c, and/or an aerosol generator.

The vaporiser 8c includes an ultrasonic piezoelectric transducer 8d for converting the source 10a of fingermark enhancement fluid 10 to vapour form.

The fingermark enhancement fluid 10 comprises a solvent, which may be aqueous or non-aqueous as required. In some embodiments the solvent may be an organic solvent, which could be a hydrofluoroether solvent, or the like. Any suitable solvent can be used, and it will be understood that the properties of the fluid transfer device 8 and the other component parts of the apparatus 1 can be designed to work with any suitable solvent.

The fingermark enhancement fluid 10 can comprise one or more reagents and/or one or more dyes, which may include at least one of: blood enhancement reagents, body fluid sensitive reagents, DNA stains or the like. The reagents could be selected from: ninhydrin, DFO, Oil Red O, ruthenium tetroxide (RTX), 1,2-Indanedione, cyanoacrylate, luminol, STK sperm tracker or the like, and the one or more dyes may be selected from BY40, R6G, RAM, Ardrox, Basic Red, or the like, and it will be apparent that other fingermark enhancement fluids 10 can be used with the apparatus 1.

The chamber 4 is configured such that a fluid flow path exists from the fluid outlet 8b of the fluid transfer device 8 to the, or each, object 6 when the object 6 is accommodated in the sealable space 4a of the chamber 4.

The chamber 4 comprises a base portion 4b, one or more walls 4c, and a top portion 4d.

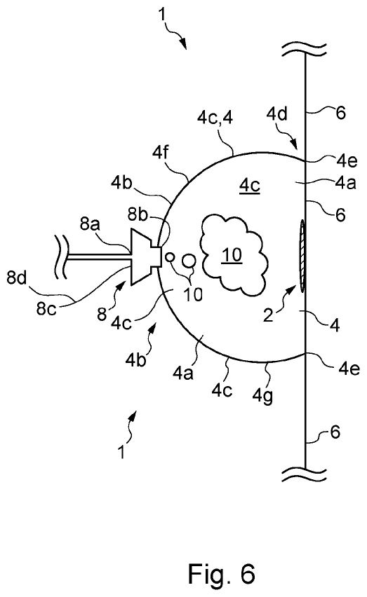

The chamber 4 is a substantially cylindrical member with a closed base portion 4b and an open top portion 4d. The chamber 4 may take any suitable shape or form, such as a cuboidal form, or as shown in the embodiment of FIG. 6, a dome shaped form. Each of the embodiments shown in FIGS. 1 to 6 are handheld and portable.

As shown in FIG. 1, and 3a to 3d, the apparatus 1 comprises a lid 12 (an example of a sealing member) for sealing the chamber 4, which forms an enclosed space when sealed by the lid 12. The lid 12 seals the chamber 4 to be substantially air-tight, but in some embodiments this may not be necessary, or a tighter seal may be required.

As shown in FIG. 2, for some objects 6, such as a wall or floor, the chamber 4 is sealable when engaged with that object 6. In this embodiment, the object 6 could be a wall, floor, or other structure/object, having a fingermark 2 thereon, and the chamber 4 could be pressed by hand against the object 6 to seal the chamber 4. The enhancement of the fingermark 2 can be carried out by vapourising the fingermark enhancement solution 10, and the chamber 4 can be removed from the wall, floor or other object 6. This is thought to be efficient, as the fingermark 2 can be processed in situ. It is advantageous that the apparatus 1 is usable in this manner by hand, as multiple areas of interest can be inspected easily and quickly.

The chamber 4 comprises one or more planar surfaces 4e at the top portion 4d thereof for engaging an object 6 comprising a fingermark 2 (shown in FIG. 2). The one or more planar surfaces 4e act as engagement members for engaging an object 6 comprising a fingermark 2, such as a wall or floor, or the like. It will be understood that other engagement members can be used. The seal formed between the chamber 4 and the object 6 is air-tight.

The chamber 4 is configurable between an unsealed and sealed configuration by either adding the lid 12 thereto, or engaging the planar surfaces 4e of the chamber 4 with an object 6. The chamber 4 is reversibly sealable between the unsealed and sealed configurations.

The fluid outlet 8b of the fluid transfer device 8 protrudes into the sealable space 4 a of the chamber 4 and is located at a mid-region 4c′ of a wall 4 c of the chamber 4.

As shown in FIGS. 1 to 3d, the apparatus 1 comprises a fan 14 (an example of a fluid flow device) configured to circulate the fingermark enhancement fluid 10 within the chamber 4. This leads to a more even distribution of the fluid 10 within the chamber 4, ensuring all objects 6 accommodated in the sealable space 4a of the chamber 4 are exposed. Whilst the fan 14 circulates the fluid 10, it will be understood that the fan 14 can disperse, or increase the flow of fluid, as required.

The fan 14 is located within the chamber 4 at a lower region 4f of the chamber 4. The fan 14 is spaced apart from the fluid outlet 8b of the fluid transfer device 8.

For the use of the apparatus 1 shown in FIG. 1 and 3a to 3d, the object 6 is typically a piece of paper or the like, whereas in FIG. 2 the object 6 is a wall/floor. The object 6 can be porous or non-porous, and the object 6 could have been pre-treated with an adhesive, such as a cyanoacrylate adhesive, as is known in the field of fingermark processing.

The chamber 4 comprises a plurality of mounting points 16 for hanging the objects 6 comprising the fingermarks 2 within the chamber 4. The fluid outlet 8b is configured to direct the fingermark enhancement fluid 10 generally towards the mounting points 16, and the fan 14 circulates the fluid 10 in the chamber 4. The mounting points 16 are located at an upper region 4g of the chamber 4.

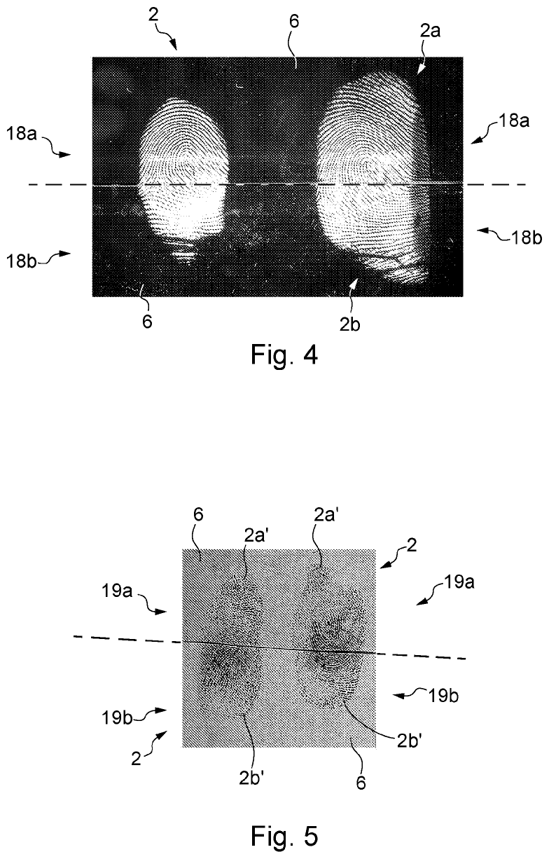

FIG. 4 shows a split image. The top half 18a of the image shows two fingermarks 2a that have been enhanced using the apparatus 1 of the invention, and the bottom half 18b of the image shows two fingermarks 2b that have been enhanced using a traditional dipping method, by immersing the fingermarks 2b in a liquid solution. Both fingermarks 2a, 2b, were enhanced using BY40, with fingermarks 2a processed using vapourised BY40, and fingermarks 2b processed using liquid BY40.

FIG. 5 shows another split image, with the top half 19a showing two fingermarks 2a′ developed using a traditional dipping method, and the bottom half 19b showing two fingermarks 2b′ processed using vaporised fingermark enhancement fluid with the apparatus 1. The fingermarks 2a′ were processed using liquid ninhydrin, and the fingermarks 2b′ were processed using vapourised ninhydrin.

FIG. 6 shows an alternative embodiment of the fingermark enhancement apparatus 1, comprising similar features to the embodiments of FIGS. 1 to 3d, denoted by the same reference numerals, with the following main differences. The chamber 4 is located at the base portion 4b. In this alternative embodiment, the chamber 4 is dome shaped, and is designed to be brought into engagement with an object 6 comprising a fingermark. The object 6 could be a wall, floor, or any object 6 comprising a fingermark. The base portion 4b of the chamber is an arcuate base portion 4b, and the walls 4c of the chamber are arcuate shaped.

In the embodiment shown in FIG. 6, the planar surfaces 4e (object engagement members) are arranged in a circular arrangement at end portions of the walls 4c of the chamber 4. The planar surfaces 4e are at the top portion 4d of the chamber 4.

Whilst the embodiment of FIG. 6 does not include a fluid flow device, it will be appreciated that this could be included within the chamber 4 shown in FIG. 6. The embodiment of FIG. 6 could also include mounting points 16, and a lid 12, as per the embodiments of FIGS. 1 to 3d.

An example of how the invention can be used will now be provided.

First, one or more objects 6 comprising a fingermark 2 are located in the sealable space 4a of the chamber 4. Alternatively, the chamber 4 is brought into engagement with an object 6 comprising a fingermark.

Next, the chamber 4 is sealed using the lid 12. This step is not required if the chamber 4 has been sealed by being engaged with an object 6.

Next, the fluid inlet 8a is connected to a source 10a of fingermark enhancement fluid 10.

The fingermark enhancement fluid 10 is provided to the chamber 4 in vapour form by vaporising the fluid 10. The fan 14 can be activated before this occurs, or during the delivery of the vapourised fluid 10 to the chamber 4, as required.

The exposure of the object 6 to the vaporised fingermark enhancement fluid 10 continues for as long as required. Then, the delivery of fluid 10 to the chamber 4 is stopped, the fan 14 is deactivated, and either the lid 12 is removed from the chamber 4, or the chamber 4 is withdrawn from the object 6.

Another object 6 can then be processed immediately thereafter.

It will be understood that the order of the steps recited in relation to the embodiments described herein could be different.

Modifications and improvements may be made to the foregoing embodiments without departing from the scope of the present invention. For example, the fluid transfer device 8 may comprise a nebuliser for nebulising the fingermark enhancement fluid, or an aerosol generator for generating an aerosol using, at least in part, the fingermark enhancement fluid 10. Any suitable components can be used to provide the fingermark enhancement fluid 10 to the chamber 4 in gaseous, vapour, or aerosol form.

In other embodiments, the chamber 4 could be opaque or non-transparent.

The apparatus 1 could comprise two or more sources 10a of fingermark enhancement fluid 10.

In the embodiments shown here, the fluid transfer device 8 comprises a single fluid inlet 8a, but other embodiments could have two or more fluid inlets 8a, the, or each fluid inlet 8a being connectable to a source of fingermark enhancement fluid 10.

The fluid transfer device 8 could comprise two or more fluid outlets 8b, the, or each, fluid outlet being configured to provide fingermark enhancement fluid 10 to the chamber 4.

The fluid transfer device 8 could be configured to provide at least one of: vapourised, aerosol, or gaseous, or combination thereof, fingermark enhancement fluid 10 to the chamber 4.

The apparatus 1 could comprise two or more fluid transfer devices 8. For example, in some embodiments, a single fluid transfer device may transfer fingermark enhancement fluid from one or more, or two or more sources thereof, to one or more fluid outlets, or two or more fluid outlets of the fluid transfer device 8. In other embodiments, two or more fluid transfer devices could be used, with any suitable configuration of fluid inlet(s) and outlet(s).

The apparatus 1 could comprise a plurality of chambers 4.

The sealing member could be a lid 12, or a seal, a hatch, a door, or the like.

1. A fingermark enhancement apparatus comprising:
a chamber comprising: a sealable space for accommodating at least a portion of an object comprising a fingermark;
a fluid transfer device for providing at least one fingermark enhancement fluid to the chamber, the fluid transfer device comprising:
a fluid inlet connectable to a source of fingermark enhancement fluid;
a fluid outlet configured to provide the fingermark enhancement fluid to the chamber;
wherein the fluid transfer device is configured to provide the fingermark enhancement fluid to the chamber in at least one of: gaseous, vapour or aerosol form.

2. The fingermark enhancement apparatus of claim 1, wherein the chamber is a portable handheld chamber and/or the fluid transfer device is a portable handheld device.

3. (canceled)

4. The fingermark enhancement apparatus of claim 1, wherein the fingermark enhancement apparatus is a portable handheld apparatus.

5. The fingermark enhancement apparatus of claim 1, wherein the fingermark apparatus is configured to be a lightweight apparatus, which is transportable by hand.

6. (canceled)

7. The fingermark enhancement apparatus of claim 1, wherein the fluid transfer device comprises a nebuliser, a vaporiser, and/or an aerosol generator.

8. (canceled)

9. The fingermark enhancement apparatus of claim 1, wherein the fluid transfer device is configured to convert the source of fingermark enhancement fluid from a liquid to at least one of: gaseous, vapour or aerosol form.

10. The fingermark enhancement apparatus of claim 1, wherein the fluid transfer device comprises a transducer for converting the source of fingermark enhancement agent to at least one of: gaseous, vapour, or aerosol form.

11. The fingermark enhancement apparatus of claim 1, wherein the apparatus comprises one or more sealing members for sealing the chamber.

12. (canceled)

13. The fingermark enhancement apparatus of claim 1, wherein the chamber is configured to be sealable when engaged with an object comprising a fingermark.

14. The fingermark enhancement apparatus of claim 10, wherein the chamber is engageable with the object by hand.

15. The fingermark enhancement apparatus of claim 10, wherein the object is a wall or other structure, having a fingermark thereon, and the chamber is pressable against the object by hand.

16. The fingermark enhancement apparatus of claim 1, wherein the apparatus is configured to disperse or circulate the fingermark enhancement fluid within the chamber.

17. The fingermark enhancement apparatus of claim 16, wherein the apparatus comprises a fluid flow device; wherein:
the fluid flow device is configured to increase the flow of the fingermark enhancement fluid within the chamber; and/or
the fluid flow device is configured to disperse, or circulate, the fingermark enhancement fluid within the chamber.

18.-21. (canceled)

22. The fingermark enhancement apparatus of claim 1, wherein the chamber comprises one or more mounting points for mounting, hanging, or locating the object(s) comprising the fingermark(s) at least partially within the chamber; and wherein the fluid outlet is configured to direct the fingermark enhancement fluid generally towards at least one of the mounting points.

23.-26. (canceled)

27. The fingermark enhancement apparatus of claim 1, wherein the fingermark enhancement fluid comprises one or more reagents and/or one or more dyes.

28.-31. (canceled)

32. The fingermark enhancement apparatus of claim 16, wherein the one or more dyes are selected from BY40, R6G, RAM, Ardrox, Basic Red.

33. A method of enhancing a fingermark, the method comprising the steps of:
(i) providing a fingermark enhancement apparatus comprising:
a chamber comprising a sealable space for accommodating at least a portion of an object comprising a fingermark;
a fluid transfer device for providing at least one fingermark enhancement fluid to the chamber, the fluid transfer device comprising:
a fluid inlet connectable to a source of fingermark enhancement fluid;
a fluid outlet configured to provide the fingermark enhancement fluid to the chamber;
wherein the fluid transfer device is configured to provide the fingermark enhancement fluid to the chamber in at least one of: gaseous, vapour or aerosol form;
(ii) locating at least a portion of an object comprising a fingermark in the sealable space of the chamber;
(iii) connecting the fluid inlet to a source of fingermark enhancement fluid; and
(iv) providing the fingermark enhancement fluid to the chamber in at least one of: gaseous, vapour or aerosol form.

34. A system for enhancing a fingermark, the system comprising:
a fingermark enhancement apparatus comprising:
a chamber comprising a sealable space for accommodating at least a portion of an object comprising a fingermark;
a fluid transfer device for providing at least one fingermark enhancement fluid to the chamber, the fluid transfer device comprising:
a fluid inlet connectable to a source of fingermark enhancement fluid;
a fluid outlet configured to provide the fingermark enhancement fluid to the chamber;
wherein the fluid transfer device is configured to provide the fingermark enhancement fluid to the chamber in at least one of: gaseous, vapour or aerosol form; and
a source of fingermark enhancement fluid.

35. The fingermark enhancement apparatus of claim 10, wherein the transducer is an ultrasonic piezoelectric transducer.

36. The fingermark enhancement apparatus of claim 27, wherein the one or more dyes includes BY40.
